(19) United States
(12) Patent Application Publication
(10) Pub. No.: US 2007/0219729 A1
(43) Pub. Date: Sep. 20, 2007

(54) System for the Evaluation of Tracer Concentration in a Reference Tissue and a Target Region

(75) Inventors: Dragos-Nicolae Peligrad, Aachen (DE); Lothar Spies, Aachen (DE); Timo Paulus, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/569,441

(22) PCT Filed: May 23, 2005
(86) PCT No.: PCT/IB05/51665

§ 371 Date: Nov. 21, 2006

(30) Foreign Application Priority Data
May 28, 2004 (EP) 04102396.1

(51) Int. Cl. G06F 19/00; A61B 5/00; G01N 33/48

(52) U.S. Cl. 702/25; 345/427; 382/131; 382/133; 382/134; 600/407; 600/410; 600/431; 600/436; 702/183

(57) ABSTRACT

The invention relates to the estimation of kinetic parameters for a target region (210) utilizing information from a normal (unperturbed) reference region (200). The proposed compartmental model models metabolic pathways for blood, reference and target tissue. The proposed analysis procedure features two alternatives: (A) Extraction of the plasma input in the reference region (200) and its unperturbed kinetic parameters, hereafter utilization of the plasma input and the kinetic parameters of the reference region as initial parameters for the analysis of the target region (210). (B) The system of kinetic equations describing the kinetics of the imaging agent in the target and reference regions is solved for the input function (amount of free imaging agent in the plasma or the total imaging agent SB(t) contained in plasma, blood elements and metabolites) considered to be the same for both regions.

The invention relates to a data processing system and a method for the evaluation of image data that represent the concentration of at least one imaging agent in a reference region and a target region of a body volume, to a record carrier on which a computer program for such an evaluation is stored, and to an examination apparatus with said data processing system.

When using medical imaging devices such as CT (Computed Tomography), MR (Magnetic Resonance), PET (Positron Emission Tomography), SPECT (Single Photon Emission Computed Tomography) or US (Ultrasound) systems to display functional or morphological properties of a patient under study, either a number of static scans or a contiguous time series of dynamic scans is recorded. To obtain the medical information of interest encoded in these images in certain applications a compartmental analysis of the underlying chemical, biological and physiological processes has to be accomplished. Compartmental analysis is based on a special type of mathematical model for the description of the observed data, in which physiologically separate pools of an imaging agent (also called tracer substance) are defined as “compartments”. The model then describes the concentration of said imaging agent in the different compartments, for example in the compartment of arterial blood on the one hand side and in the compartment of tissue on the other hand side (it should be noted, however, that in general compartments need not be spatially compact or connected). Typically, there is an exchange of substance between the various compartments that is governed by differential equations with (unknown) parameters like exchange rates. In order to evaluate a compartment model for a given observation, the differential equations have to be solved and their parameters have to be estimated such that the resulting solutions optimally fit to the observed data. More details on the technique of compartmental analysis may be found in the literature (e.g. S. Huang and M. Phelps, “Principles of Tracer Kinetic Modeling in Positron Emission Tomography and Autoradiography” in: M. Phelps, J. Mazziotta, and H. Schelbert (eds.), Positron Emission Tomography and Autoradiography: Principles and Applications for the Brain and Heart, pp 287-346, Raven Press, New York, 1986).

In dynamic compartmental analysis the so-called “(plasma) input function” defines the amount of imaging agent (free and/or metabolized) within the blood which can go into the tissue. It cannot be easily determined non-invasively. In order to circumvent the need to measure the input function invasively (by drawing venous or arterial blood samples), the dynamic analysis sometimes utilizes the reference tissue concept, wherein the total time signal curves (TSC) are detected in two different tissue regions (VOIs) called “reference tissue” and “target tissue” (cf. J. S. Perlmutter, K. B. Larson, M. E. Raichle, J. Markham, M. A. Mintum, M. R. Kilbourn, M. J. Welch: “Strategies for In Vivo Measurement of Receptor Binding using Positron Emission Tomography”, J. Cereb. Blood Flow Metab. 6, (1986) pp 154-169; M. Ichise, J. H. Meyer, Y. Yonekura: “An Introduction to PET and SPECT Neuroreceptor Quantification Models”, Jour. of Nucl. Med. 42, (2001) pp 755-763 with more references). For this concept it is assumed that the input function to both tissues (reference and target) is the same. Furthermore, the following assumptions need to be imposed: Firstly, no metabolic products are produced within the tissues which may get washed out, and secondly, metabolic products cannot penetrate the blood-tissue barrier. Thus since the input function is substituted by the time signal curve (TSC) of the reference tissue, the reference tissue TSC will act as a “filter” for the metabolic products within blood. Nevertheless all these assumptions are legitimate only if the fractional blood volume within the observed region is negligible small, and if there is no binding (within the tissue of interest, red cells, platelets or plasma protein) of the imaging agent in the reference region and additionally if no penetration of the metabolic products through the blood-tissue barrier is possible. Due to the limited spatial resolution of the PET scanners, however, VOIs may in practice contain not only tissue but also blood elements and metabolites, which can in general penetrate the blood-tissue barrier. Both the amount of free (unmetabolized) imaging agent and labeled metabolites in plasma can be assayed directly from samples of blood taken throughout the detection (scan) whereas the contribution of labeled metabolites to the tissue signal cannot be measured directly. All this may contaminate the results derived with state-of- the-art techniques, which suffer from the limitations just mentioned.

Based on this situation it was an object of the present invention to provide means for a more realistic evaluation of a reference region and a target region of image data.

This object is achieved by a data processing system according to claim 1, a record carrier according to claim 10, an examination apparatus according to claim 11, and a method according to claim 12. Preferred embodiments are disclosed in the dependent claims.

The data processing system according to the invention serves for the evaluation of image data that represent the concentration of at least one imaging agent in a reference region and a target region of a body volume. The image data may for example be PET scans that represent the spatial distribution of a radioactive imaging agent. The data processing system may particularly be realized by a microcomputer comprising usual components like (volatile or nonvolatile) memory, processors, I/O interfaces and the like together with the necessary software. The data processing system is adapted to evaluate a (composite) compartment model of the reference region and the target region, the model comprising compartments which account for
Free imaging agent in blood or parts thereof (e.g. plasma, blood elements).
Imaging agent that is specifically bound in the target region, wherein the “specific binding” is that kind of binding which is in the focus of interest of the investigation.
Imaging agent that is nonspecifically bound in the target region, the reference region and/or in blood (particularly in blood elements).
Imaging agent that is present in metabolites or other trapping systems.

The aforementioned data processing system allows a very precise and realistic evaluation of image data because it applies a compartment model for both a reference and a target region which comprises compartments that account for the principal contributions of the imaging agent to the measured signals. Particularly, the model accounts for a fractional volume of blood that is present in the observed regions and for metabolites of the imaging agent. In both cases, the bound/metabolized imaging agent contributes indistinguishably from free imaging agent to the measured signal (for example radioactivity) while at the same time behaving differently with respect to the physiological processes that shall be observed. The accuracy of the examination will therefore be improved if such background signals are known and can be compensated.

According to a first further development of the invention (called “method A” in the “Description of preferred embodiments” below), the data processing system is adapted to evaluate in a first step the compartment model for the reference region, and to evaluate in a second step the compartment model for the target region based on the results of the first step. This procedure makes use of the fact that the reference region and the target region are normally only coupled via the blood. Therefore, the reference region may be evaluated first and separate from the target region. Moreover, the target region is typically very similar to the reference region besides the specific binding processes that shall be investigated. Results obtained for the reference region may therefore be used in the second step during the evaluation of the (more complex) target region.

In a preferred embodiment of the aforementioned data processing system, the free imaging agent in plasma is determined in the first step of the evaluation procedure and taken as input function for the evaluation process of the target region in the second step. The amount of free imaging agent in blood (plasma and/or blood elements) is an important value that must be known for the evaluation of compartment models which describe the uptake of imaging agent from blood. The proposed determination of this amount by the evaluation of the reference region allows to obtain this information without drawing blood samples from a patient.

According to another embodiment of the data processing system described above, the model parameters for the reference region (like transfer rates) that were calculated in the first step of the evaluation procedure are taken as starting values for the corresponding model parameters of the target region in the second step. This approach exploits two features that are typically present: firstly the similarity of the compartment models for the reference region and the target region (which differ only in a few components that describe the specific binding of the imaging agent), and secondly the fact that the specific binding in the target region has only little influence on the properties of the other components, thus allowing to transfer the results from the reference region to the target region (“perturbation assumption”).

According to a second further development of the invention (called “method B” in the “Description of preferred embodiments” below), the data processing system is adapted to solve the system equations that belong to the considered compartment model for the target region and the reference region simultaneously based on the assumption that both regions have the same input function. This input function may particularly be the free imaging agent contained in plasma or the total imaging agent contained in blood, wherein the total imaging agent comprises free imaging agent and bound imaging agent (for example in blood elements and metabolites). The aforementioned assumption reflects the fact that the reference and target region are coupled to the same pool of blood and that (due to their vicinity) the conditions in the blood fractions contained in both regions are approximately the same.

According to a further development of the invention, the data processing system is adapted to compare evaluation results for corresponding components of the reference region and the target region in order to validate the correctness of the approach. As was already mentioned, the specific processes in the target region have only little influence on the other processes of the target region, wherein the latter processes do similarly exist in the reference region. Therefore, the results for components which are present both in the reference region and the target region should be similar to each other, and the conditions in the target region may be considered as a perturbation of the conditions in the reference region. A violation of this assumption then indicates that the underlying approach to analyze the regions, particularly the chosen compartment model, may not be optimal and should be replaced by a better one.

According to a further development of the invention, the data processing system is adapted to calculate errors that are associated with the evaluation of the image data based on different compartment models. Thus, various compartment models with for example different numbers of compartments may be applied to the measured image data and evaluated with respect to said error. A comparison of the resulting errors then allows to select a model that seems to be most appropriate for the description of the measurements.

The processing system may particularly comprise a display unit on which the results of the evaluation procedures may be displayed. The graphical display of the available information (times signal curves, parametric maps, morphological information etc.) is an important aspect of the data processing system as it allows a physician a fast, intuitive access to the available information.

The invention further comprises a record carrier, for example a floppy disk, a hard disk, or a compact disc (CD), on which a computer program for the evaluation of image data that represent the time varying concentration of at least one imaging agent in a reference region and a target region of an object is stored, wherein said program is adapted to evaluate a compartment model of the reference region and the target region with compartments accounting for imaging agent that
(i) is free in blood or parts thereof;
(ii) is specifically bound in the target region;
(iii) is unspecifically bound in the target region, reference region and/or in blood (particularly in blood elements);
(iv) is present in metabolites or other trapping systems.

Moreover, the invention comprises an examination apparatus with an imaging device for generating image data that represent the time varying concentration of at least one imaging agent in an object, and a data processing system of the kind described above. The imaging device may for example be a PET, SPECT, CT, MR, or US system.

Finally, the invention comprises a method for the evaluation of image data that represent the distribution of at least one imaging agent in a reference region and a target region of a body volume, comprising the evaluation of a compartment model of the reference region and the target region with compartments accounting for imaging agent that
(i) is free in blood or parts thereof;
(ii) is specifically bound in the target region;
(iii) is unspecifically bound in the target region, reference region and/or in blood (particularly in blood elements);
(iv) is present in metabolites or other trapping systems.

The aforementioned record carrier, examination apparatus, and method rely on the features of a data processing system as it was described above. For more information on details, advantages and further developments of the record carrier, examination apparatus, and method, reference is therefore made to the description of the data processing system.

These and other aspects of the invention will be apparent from and elucidated with reference to the embodiment(s) described hereinafter.

In the following the invention is described by way of example with the help of the accompanying drawings in which:

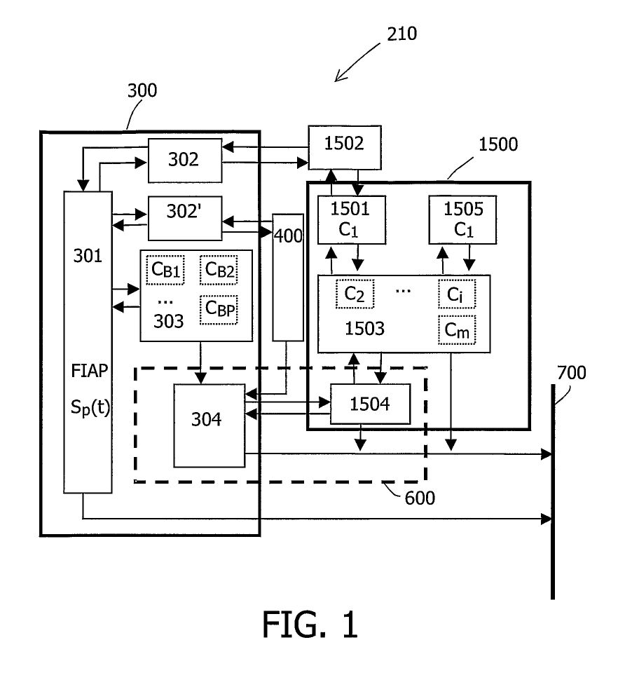

FIG. 1 shows a compartment model of the target region according to a first evaluation procedure called “method A”;

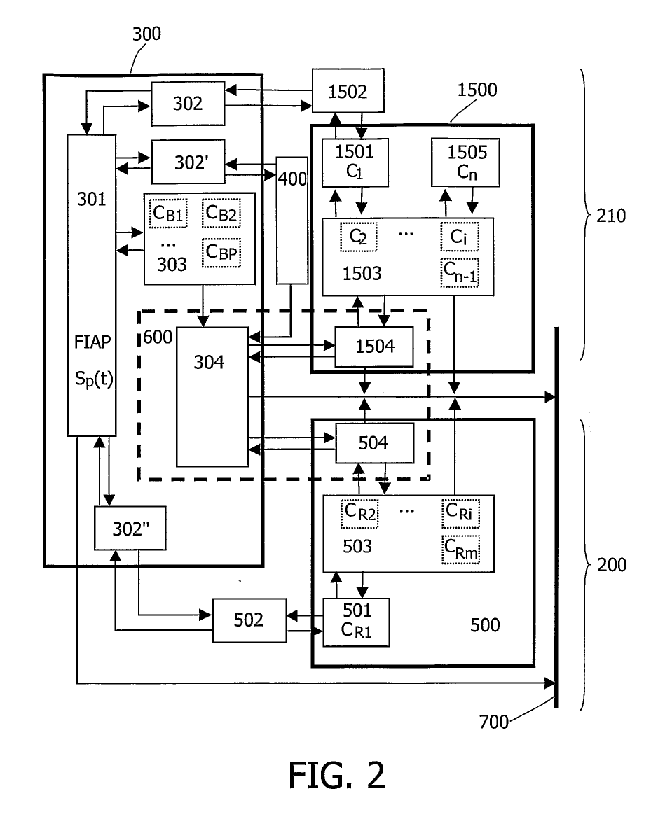

FIG. 2 shows a compartment model of the target region and the reference region according to a second evaluation procedure called “method B” when the system of ODE is solved for the free imaging agent in plasma;

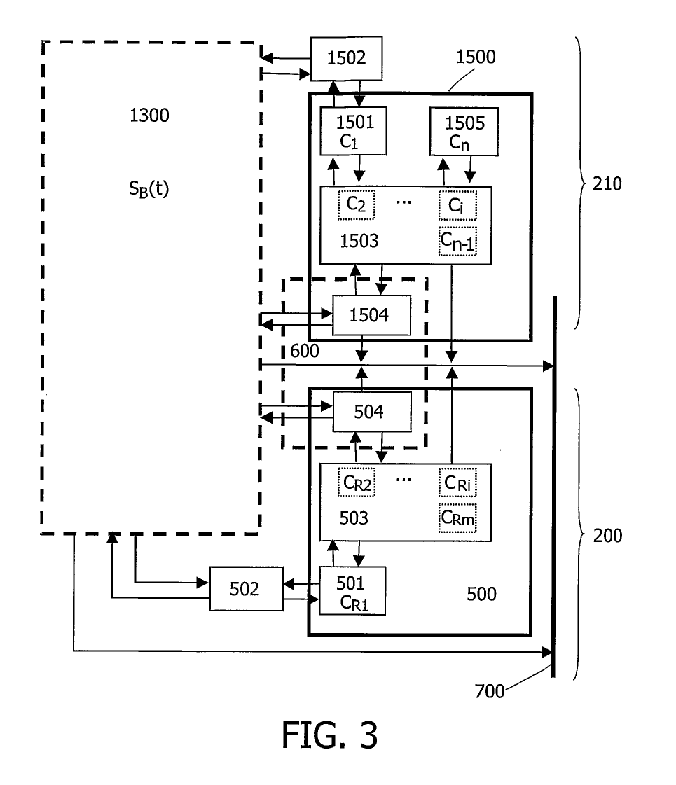

FIG. 3 shows the compartment model of the FIG. 2 when the system of ODE is solved for the entire amount of imaging agent in blood;

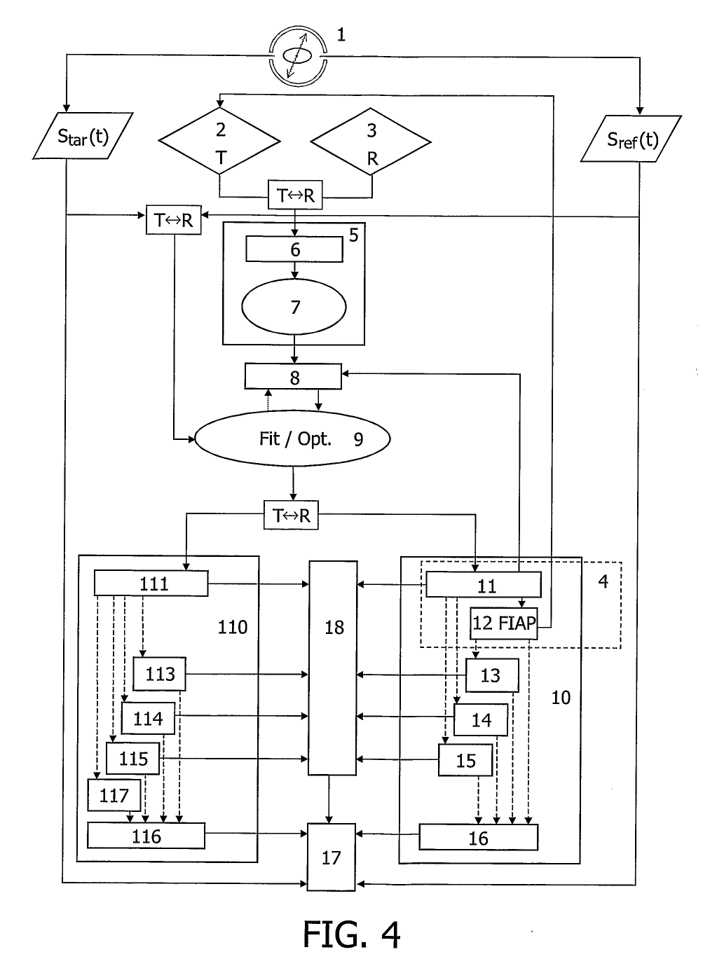

FIG. 4 is a flowchart of the kinetic analysis of the reference and target regions according method A, assuming as input the free imaging agent in plasma (FIAP);

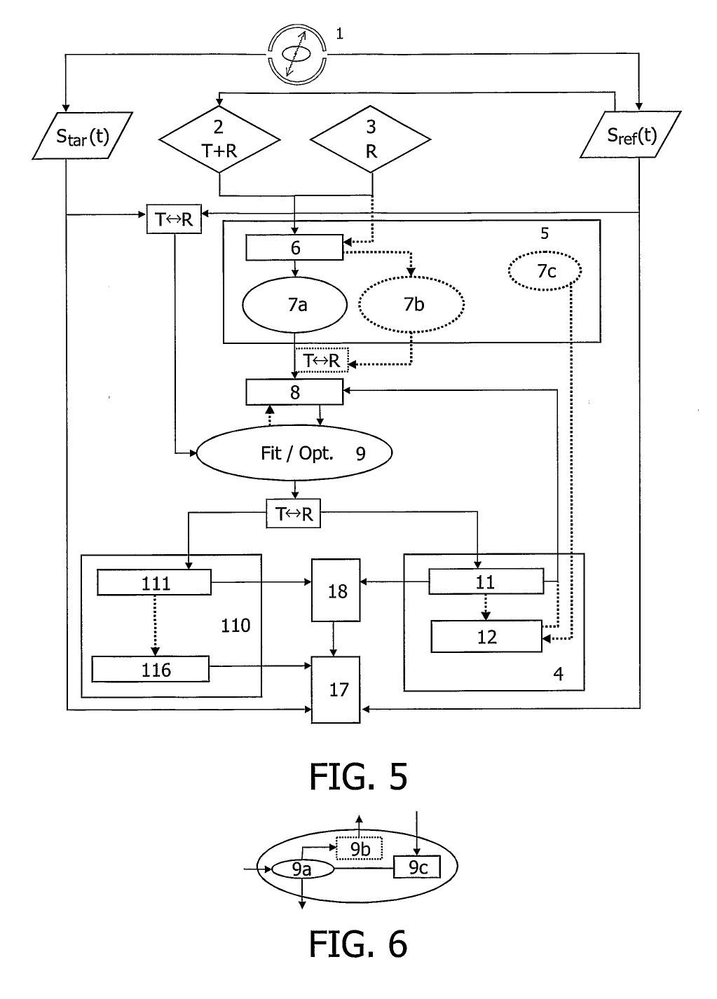

FIG. 5 is a flowchart of the kinetic analysis of the reference and target regions according method B, assuming as input the total signal from the imaging agent within the reference region.

FIG. 6 is a particular embodiment of the “Fit and Optimization” block 9 of FIGS. 4 and 5.

In the Figures, like numerals correspond to like components. In FIGS. 2 and 3, the numerals of components of the target tissue 1500 correspond to those of similar components of the reference tissue 500 plus 1000.

In the following a compartmental analysis and quantification of a reference region and a target region based on a composite compartmental model will be described that is suitable for clinical studies and that takes all shortcomings mentioned in the section “Background of the invention” into account: fractional blood volume, metabolites that cross the blood-tissue barrier and/or other unspecific binding sources within the tissues. To take advantage of the maximal resolution which the imaging devices can supply, this analysis has to be done on a per-voxel basis. In order to perform the full dynamic analysis a set of assumptions and concepts are introduced and discussed below.

The basic idea is to propose a general composite compartmental model (topology) and dynamic analysis procedure to extract the kinetics of the imaging agent within target and reference regions or volumes of interest (VOIs). The composite compartmental model contains subsystems which have compartments that account for the amount of imaging agent distributed in both the target and reference regions either as free (umnetabolized) in the plasma, or bound in tissue (target and reference), blood elements (like red cells, platelets, plasma protein etc.), metabolites and/or other trapping sources within the VOIs under study (see FIGS. 1-3). The proposed procedure for analysis consists either in the dynamic analysis of the target region using the input function expressing the local free (unmetabolized) imaging agent concentration in plasma (FIAP) decomposed from measurements of the reference region, or, alternatively, in solving the system of kinetic equations describing the target and reference regions for the unknown input function assumed to be the same for both regions. In each VOI, the detection is represented by time signal curves (TSCs). Thus the model is suited for cases where the contribution from the blood volume fraction (which contains the FIAP) and metabolite TSCs cannot be accurately separated from the total tissue TSC. The procedure is applicable to either the complete set of recorded images (either several static scans or the 4-dim. time-scan data based on a VOI analysis, images which can be reconstructed images from a PET, SPECT, MRI or US scanner) and generates from the input maps of all the relevant chemical, biological and physiological parameters on a per-voxel basis.

The following concepts and definitions are used in the embodiment to be described:

It will be distinguished between specific or nonspecific binding of the labeled imaging agent within either diffusible (“delocalized”) or non-diffusible (“localized”) binding sites. Here specific binding means targeted binding (the kind of binding that shall be investigated) whereas the nonspecific binding will contribute to the “background” considered as the total amount of detectable imaging agent which does not participate in the specific binding process. For the sake of generality the labeled imaging agent should not be considered always as detectable in order to include also the smart imaging agents. Often the binding process is the metabolization of the imaging agent within the blood or tissues under study. The labeled metabolized imaging agent binds either specifically or unspecifically to non-diffusible binding sites (tissue or blood elements like red cells, platelets, plasma protein etc.) or to diffusible binding sites which can freely circulate the entire body as labeled metabolites.

In the “non-specific binding” subsystems (regions) within the VOI the imaging agent can flow freely directly or indirectly from plasma into these regions, move freely among compartments of these subsystems, and, in turn, flow back into the plasma. These subsystems are called reversible since the imaging agent transfer between and among the plasma and those subsystems is fully reversible. Usually the imaging agent in the unspecific binding subsystems (regions) leaves this part of the system by going into either the plasma (if unmetabolized) or (if metabolized) into other subsystems which can be freely diffusible and/or act as specific binding sites.

A special case is when the in- and efflux are equal over the detection time frame such that the amount of circulated imaging agent is conserved. In this case the imaging agent is absent at both the initial moment and the end of the detection. Such regions are called “free” or “loss-less” since the imaging agent returns unchanged in the plasma. For sake of completeness one should mention that the unspecific binding tissue regions may not rapidly equilibrate with the regions where the imaging agent is free as usually assumed. Usually the transfer processes of the imaging agent within the system obeys first-order linear or nonlinear kinetics.

The “specific binding” subsystems (regions) within the VOI are called irreversible since the imaging agent after entering this region from the plasma and/or reversible tissue regions cannot leave the binding site within the detection time frame, neither back to plasma nor to the reversible tissue regions. The irreversible subsystems consist of one or more compartments that can always be mathematically lumped together into a single compartment called “uptake”. In real systems the transfer process is not completely irreversible and it is usually described by reversible compartments having only very slow efflux (loss) of imaging agent entering either the plasma or another reversible part of the system.

In the “trapping” subsystems (regions) within the VOI, also called “lossy”, the labeled imaging agent can flow-in freely directly or indirectly from plasma but cannot flow back into the plasma since it will be irreversible non-specifically bound within these subsystems. Furthermore the “trapping sources” may not be—but are often—freely diffusible, i.e. they can circulate the entire body, can flow in and out and/or move freely among reversible subsystems other then the trapping one. Therefore the signal measured from these “lossy” tissue regions will contribute also to the overall detection (total tissue signal) and in most of the cases it will contaminate the signal detected from the specific binding of imaging agent in the target tissue.

The following general assumptions are made for the transport model illustrated in FIG. 1 (method A) and FIGS. 2 and 3 (method B):

Assumptions for the imaging agent:
i. The imaging agent, which may for example be F-MISO (F-Fluoromisonidazole), is assumed to be delivered via arterial blood flow and transported into tissue by active/mediated transport diffusion. There is a single source, namely the free imaging agent in the plasma (FIAP) 301, its concentration being denoted as Sₚ. In practice there may be cases where more than one imaging agent is used or where more than one input is considered. Nevertheless these generalizations will not affect the proposed procedure for analysis in its main concepts and can be covered by a straightforward extension thereof.
ii. The imaging agent does not perturb (alter) the system and is not initially present in the tissue regions (either reversible or irreversible).
iii. The extraction fraction of the free imaging agent from plasma into tissue is not necessarily small and thus the rate of transport to tissue can be dependent on blood flow (see panel 502, 1502 “Perfusion/Extraction”). For the sake of generality also the dispersion representing the different “biochemical” distances needed for the free imaging agent from the moment of injection until it starts supplying the target and reference tissue should be considered (see panels 302, 302′, 302″ “Dispersion”).

Assumptions for the role of labeled metabolites within the system:
iv. As already mentioned one of the major problems in the quantitative interpretation of dynamic scans is the presence of detectable labeled metabolites. In this example it will be assumed that all labeled metabolites formed in the system under study are detectable and contribute to the total tissue signal in the sense of a contamination of the data. Labeled metabolites in tissue may result either from the metabolism of the free imaging agent within the blood elements and tissues under study or be taken up from the blood during the detection (scan). It is possible to extend this analysis also for cases where various types of metabolites are labeled with different types of imaging agents in order to distinguish their contribution to the total tissue signal. In this case a separate detection using multiple scans is necessary (cf. S. C. Huang, J. R. Barrio, D. C. Yu, B. Chen, S. Grafton, W. P. Melega, J. M. Hoffman, N. Satyamurthy, J. C. Mazziotta, M. E. Phelps: “Modelling approach for separating blood time-activity curves in positron emission tomographic studies”, Phys. Med. Biol., 36, (1991) pp 749-761). In such cases the actual procedure for analysis should be applied for each type of imaging agent separately.

Most of the metabolites in the blood supplying the tissue (see panel 304 “Metabolites in Blood”) are either due to the peripheral metabolism (see panel 400 “Organs”) or blood metabolism of the free imaging agent (panel 303 “Blood Elements”). In blood there may also be an uptake of metabolites formed from the free imaging agent passing through either interstitial or intracellular space within the tissue of study. As stated earlier, one distinguishes between the metabolized imaging agent either specifically bound to target binding sites or non-specifically bound to diffusible and/or non-diffusible binding sites within the VOI. The detectable metabolized imaging agent bound to diffusible binding sites leaves the tissue regions and flows back into the blood but it is no longer available as free imaging agent for further possible metabolization processes, i.e. it can penetrate the blood-tissue barrier again only as metabolites. Thus for the sake of generality one can consider as “metabolites” all the trapping sources of the system under study, i.e. the amount of imaging agent irreversible and non-specifically bound within the VOI. In conclusion the role of metabolites within the system can be described by an appropriate trapping subsystem (see dashed panel 600 “Metabolites”) which can contain one or more compartments (e.g. within blood, tissue or reference tissue) which can be mathematically lumped together in a common metabolite pool if the assumption is valid that the metabolites produced within tissues (interstitial or intracellular for target and reference tissue) or in blood elements will exchange rapidly with the metabolites in the blood. Metabolites (as well as free imaging agent from 301) may leave the body permanently to the “Exit” 700.
v. In this example the case is considered that the metabolites within the blood can penetrate through the blood-tissue barrier in any kind of tissue regions, containing any kind (specific and/or nonspecific) of binding sites.
vi. Moreover, the clearance of the labeled metabolites out of the body either from the blood metabolite pool or directly from the tissues under study is considered.

Assumptions for the labeled blood elements in the VOI:
vii. The blood elements consist of a subsystem having compartments which communicate reversibly only with the free imaging agent in the plasma (see panel 303 “Blood Elements”). The reversible communication with the plasma can occur directly or indirectly through intermediary compartments which can all be mathematically lumped together only if they rapidly equilibrate during detection of the amount of free imaging agent in plasma. It will be assumed in this example that all the labeled blood elements within the blood are detectable and contribute to the total tissue signal in the sense of a contamination of the data.
viii. The blood elements can metabolize the free imaging agent within the blood as mentioned already.
ix. The blood elements cannot pass the blood tissue barrier and should not be allowed to diffuse into the tissue regions of the VOI.

Assumptions for the reference tissue 500:
x. The reference tissue 500 may consist of a number of subsystems each of them having compartments which communicate reversibly with the free imaging agent in the plasma. This reversible communication with the plasma can occur directly or indirectly through intermediary compartments. There is at least one compartment 501 (e.g. the interstitial) in which the imaging agent within the tissue is considered as “free”. This compartment can be mathematically lumped together with other nonspecific binding compartments if the amount of transferred imaging agent between the compartments rapidly equilibrates during detection. The interstitial compartment models the properties of the tissue membrane.
xi. The reference tissue 500 is not specifically binding the imaging agent; therefore it should not contain any irreversible subsystems.
xii. All types of metabolites can flow in and out of the reference tissue 500 but they cannot be irreversible bound within. Also no metabolization of the free imaging agent within the reference tissue should be allowed. Thus a free movement of the metabolites among all reversible subsystems other than the interstitial ones is not allowed (i.e. the metabolites subsystem has no exchange with any other reversible subsystem except the interstitial one).
xiii. Finally, no oscillation of the free imaging agent within the entire system under study should be mathematically allowed since such oscillatory processes are physiologically extremely improbable.

In order to achieve a minimum of contamination and thus reliable quantification the best candidates for the reference tissue are therefore homogeneous regions where the vascularization and nonspecific binding of the imaging agent within it are minimal.

Assumption for the target region 210:
xv. The target region 210 or VOI should consist of a number of nonspecific binding subsystems each of it having compartments which communicate reversibly with the free imaging agent in the plasma. The reversible communication with the plasma can occur directly or indirectly through intermediary compartments. As in the case of the reference region, there is at least one compartment 1501 (e.g. the interstitial) in which the imaging agent within the tissue is considered as “free”.
xvi. The target region can specifically bind the imaging agent and therefore it does contain irreversible compartments 1505 which depending on the type and sensitivity of the detection and on the choice of the VOIs can be mathematically reduced (by diagonalization of the system matrix) into a single uptake compartment.
xvii. In most cases the target region can also metabolize the free imaging agent. Thus a trapping subsystem 1504 containing both the amount of imaging agent metabolized within the tissue (both interstitial or intracellular origin) and the metabolites which penetrate reversible into the tissue from the plasma have to be considered. This subsystem should communicate reversibly with the nonspecific binding subsystem 1503 and with the amount of metabolites in the plasma (304). The trapping subsystem 1504 in the tissue can be mathematically lumped together with the trapping subsystem 304 in the blood if a rapid equilibration between the metabolites in tissue and plasma is established.
xviii. Similar to the case where the reference tissue was considered, no oscillation of the free imaging agent within the entire system under study should be mathematically allowed.

In preparation of the dynamic analysis methods according to the present invention, a perturbation concept is explained in the following that contains further assumptions with respect to the target and reference region.

Define as “unperturbed” a fictive target region VOI in which the imaging agent is neither specifically bound nor metabolized in any of its volume fractions containing blood or tissue. The kinetics of the imaging agent is then given by regions containing no or only unspecific binding sites. If due to some process in one of the volume fractions within this target region (for example in the tissue) the imaging agent starts to be specifically bound and metabolized (specifically and/or non-specifically), the kinetics in said target region will change. The specific binding and metabolization can be considered as a perturbation of the unperturbed fictive target tissue. One may now make the assumption that the perturbation will influence only slightly the kinetics of the imaging agent in the other parts of the volume fraction, i.e. in regions where the agent can be free (unmetabolized) or non-specifically bound. It is important to note that the perturbation itself should not be considered small, but only its influence upon the kinetics of the imaging agent in the surrounding regions where it is free or non-specifically bound should be considered in a first order approximation as small. In other words the correlation of the kinetic parameters describing the kinetics within the regions where the imaging agent is specifically bound or metabolized with the kinetic parameters describing the kinetics within regions where the agent is either free or non-specifically bound should be in a first order approximation small. Thus the perturbation condition can be formulated as follows:

All perturbation sources within the target region like the specific binding subsystems or subsystems where the imaging agent is metabolized (specifically and/or non-specifically) will produce only a small perturbation of the kinetics in the subsystems where the imaging agent is either free or non-specifically bound within the same target region.

Thus the dynamic parameters describing the kinetics within subsystems of the target region where the imaging agent is either free or non-specifically bound are considered as perturbed with respect to the unperturbed parameter values characterizing the kinetics of the imaging agent in the same target region in the absence of the perturbation. Additionally the kinetics of the imaging agent in the blood will be practically not influenced by the metabolization processes (and thus the specific binding) within the target region since it is determined mainly by the peripheral metabolism in the body organs. Therefore the perturbation within the target tissue volume fraction should have practically no influence upon the kinetics of the imaging agent in the blood volume fraction within the same target region VOI. Finally since the fictive unperturbed target region is not measurable, the “reference tissue condition” should be applied in order to extract the unperturbed dynamic parameters. According to the “reference tissue condition” it is assumed that the kinetic properties of the free and nonspecifically bound imaging agent transport in the fictive unperturbed target tissue VOI resemble at any moment of time those in the free and nonspecific binding subsystems of the reference tissue. Therefore the dynamic parameters (including the steady state values, e.g. volumes of distribution) of the reference tissue can be considered as unperturbed. Moreover, the dynamic parameters describing the kinetics within the blood volume fraction and the free and/or non-specifically binding tissue subsystems within the target region VOI should not vary significantly from those obtained from the analysis of the reference region.

In order to circumvent the need to measure invasively the arterial input function, two methods are proposed and explained in the following.

Method A:

First a set of “unperturbed dynamic parameters” (e.g. transport and metabolism rates) available from the reference region analysis are used as input parameters for the analysis of the target region having as input-function the free amount of imaging agent in plasma, Sₚ, extracted by decomposition of the total reference region signal. This implies the perturbation assumption to be valid, i.e. that the above mentioned input-function is the same for both target and reference region and that the dynamic parameters in the target region (including fractional blood and metabolites) will differ only slightly in all free and unspecific binding subsystems (in the sense of a small perturbation) from the unperturbed parameters obtained in the same kind of subsystems within the reference region. Since the input function and the full set of unperturbed dynamic parameters have to be known in order to describe the detected total time signal, a complete dynamic analysis of the reference region is presumed (a more detailed discussion will follow below).

Method B:

Secondly one can solve the system of kinetic equations describing the dynamics of the imaging agent in the target and reference regions for the input function considered to be the same for both tissue regions or VOIs. This input function can be either the amount of free imaging agent in the plasma, S_{P}(t), (cf. FIG. 2.) or the total imaging agent (free and metabolized), S_{B}(t), contained in the entire blood volume fraction 1300 (subsystem) including plasma, blood elements and metabolites (cf. FIG. 3.). This implies either to solve the equations

$$\{ \begin{matrix}S ( t ) = \{ \begin{matrix}{V}_{B} ( [ 1 - ( α + β ) ] δ ( t ) + α {G}_{B} ( t ) + \\ β {G}_{MB} ( t ) ) + {V}_{T} γ {G}_{MT} ( t ) + {V}_{T} {G}_{T} ( t )\end{matrix} \} ⊗ {S}_{P} ( t ) {V}_{B}^{- 1} \\ {S}_{0} ( t ) = \{ \begin{matrix}{V}_{B 0} ( \begin{matrix}[ 1 - ( {α}_{0} + {β}_{0} ) ] δ ( t ) + \\ {α}_{0} {G}_{B 0} ( t ) + {β}_{0} {G}_{MB 0} ( t )\end{matrix} ) + \\ {V}_{T 0} {γ}_{0} {G}_{MT 0} ( t ) + {V}_{T 0} {G}_{T 0} ( t )\end{matrix} \} ⊗ {S}_{P 0} ( t ) {V}_{B 0}^{- 1}\end{matrix} \qquad ( 1 )$$

for the same input C_{P}(t)\textcircled{x}S_{INPUT}(t)=S_{P}(T)/V_{B}=S_{P0}(T)/V_{B0}, or to solve the equations

$$\{ \begin{matrix}S ( t ) = \{ {V}_{B} δ ( t ) + {V}_{T} γ {G}_{MT} ( t ) + {V}_{T} {G}_{T} ( t ) \} ⊗ {S}_{B} ( t ) {V}_{B}^{- 1} \\ {S}_{0} ( t ) = \{ {V}_{B 0} δ ( t ) + {V}_{T 0} {γ}_{0} {G}_{MT 0} ( t ) + {V}_{T 0} {G}_{T 0} ( t ) \} ⊗ {S}_{B 0} ( t ) {V}_{B 0}^{- 1}\end{matrix} \qquad ( 2 )$$

for the input function assumed to be

$$\begin{matrix}{S}_{B} ( t ) / {V}_{B} = ( [ 1 - ( α + β ) ] {C}_{P} ( t ) + α {G}_{B} ( t ) + β {G}_{MB} ( t ) ) ⊗ {S}_{INPUT} ( t ) \\ ≅ ( [ 1 - ( {α}_{0} + {β}_{0} ) ] {C}_{P} ( t ) + {α}_{0} {G}_{B 0} ( t ) + {β}_{0} {G}_{MB 0} ( t ) ) ⊗ {S}_{INPUT} ( t ) \\ = {S}_{B 0} ( t ) / {V}_{B 0}\end{matrix}$$

Here G_{T}(t), G_{MT}(t) are the impulse-response functions of the tissue and metabolites subsystems within the tissue volume fraction V_{T}, whereas G_{B}(t), G_{MB}(t) represent the impulse-response of the blood elements and metabolites subsystems within the blood volume fraction V_{B} of the same target region. α and β are the partial volume fractions of the blood elements and the metabolites, respectively, within the blood subsystem and in the appropriate target and/or reference tissue ROI. Similarly γ is the partial volume fraction of the metabolites within the target and/or reference tissue ROI. S_{INPUT}(t) is the input function of the imaging agent which can be for example either a known injection function (which describes the injection of the imaging agent into the body by for example the volume flow of imaging agent through a syringe; Method A) or which is considered as unknown as in Method B. Subscripts T stands for “tissue”, B for “blood”, MB for metabolites within blood, MT for metabolites within tissue, and index “0” for unperturbed (i.e. reference) region. C_{P}(t) is the concentration of the free imaging agent in plasma. The tissue volume fraction V_{T} may comprise both interstitial and intracellular volume fractions. The impulse-response functions are the solution of a system of ordinary differential equations (ODE) associated to the compartmental topology particularly considered (as in FIGS. 1-3), wherein the δ(t) -function is taken as input. Finally the total target region signal S(t) can be expressed in terms of the total reference region signal S₀(t) (considered as input) following the general composite solution for the detected total time signal expressed as

$$\begin{matrix}S ( t ) = {L}^{- 1} \{ \frac{[ \begin{matrix}{V}_{B} ( [ 1 - ( α + β ) ] + α {Γ}_{B} ( s ) ) + \\ {V}_{B} β {Γ}_{MB} ( s ) + {V}_{T} γ {Γ}_{MT} ( s )\end{matrix} ] + {V}_{T} {Γ}_{T} ( s )}{\begin{matrix}{V}_{B 0} ( [ 1 - ( {α}_{0} + {β}_{0} ) ] + {α}_{0} {Γ}_{B 0} ( s ) ) + \\ {V}_{B 0} {β}_{0} {Γ}_{MB 0} ( s ) + {V}_{T 0} {γ}_{0} {Γ}_{MT 0} ( s ) + {V}_{T 0} {Γ}_{T 0} ( s )\end{matrix}} \} ⊗ {S}_{0} ( t ) \\ when the free imaging agent in plasma was \\ replaced ( cf . FIG . 2 , Eq . ( 1 ) ) . \\ Alternatively , a simplified composite solution & ( 3 ) \\ S ( t ) = {L}^{- 1} \{ \frac{[ {V}_{B} + {V}_{T} γ {Γ}_{MT} ( s ) ] + {V}_{T} {Γ}_{T} ( s )}{{V}_{B 0} + {V}_{T 0} {γ}_{0} {Γ}_{MT 0} ( s ) + {V}_{T 0} {Γ}_{T 0} ( s )} \} ⊗ {S}_{0} ( t ) & ( 4 )\end{matrix}$$

holds for the case when the total imaging agent in the whole blood subsystem was replaced (cf. FIG. 3, Eq. (2)). Here Γ_{T0}, Γ_{B0}, Γ_{MB0}, Γ_{MT0} are the Laplace-(L)-transformed impulse-response functions (Γ=LG) describing the kinetics in all subsystems of the reference region, whereas Γ_{T}, Γ_{B}, Γ_{MB}, Γ_{MT} are the Laplace-transformed impulse-response functions describing the target region. Equations (3) and (4) contain comprehensively all the dynamic parameters describing the kinetics of the imaging agent in the complete system under study, i.e. in the target and reference region including also the corresponding blood elements and metabolites subsystems. Finally, the inverse Laplace-transformation can be easily evaluated analytically or numerically depending on the specific choice of the compartmental topology.

Comparison of Methods A and B:

The kinetic processes (detection) of the imaging agent in the target and reference region VOIs described comprehensively in the system from Eq. (1) are physiologically independent of each other. Nevertheless the equations in Eq. (1) can be determined either mathematically independent of each other (decoupled) as in method A or coupled as in method B since the input function of the target region is expressed with the kinetic parameters of the reference region. If solved analytically, the working formula for method A is given by

$$S ( t ) = \{ \begin{matrix}{V}_{B} ( [ 1 - ( α + β ) ] δ ( t ) + α {G}_{B} ( t ) + β {G}_{MB} ( t ) ) + \\ {V}_{T} γ {G}_{MT} ( t ) + {V}_{T} {G}_{T} ( t )\end{matrix} \} ⊗ {C}_{P} ( t ) \qquad ( 5 )$$

It is mathematically much simpler than the form obtained from Eqs. (3) and (4) because it depends only on the impulse-response functions of the subsystems contained within the target region, i.e. only on the dynamic parameters describing the kinetics of the imaging agent in the target region under study. For similar reasons the dynamic parameters describing the kinetic in the reference region enter in Eq. (5) only as known parameters into the input function expression. Thus the elements of the Jacobian matrix (i.e. the partial derivatives with respect to the dynamic parameters) will also be much simpler than the corresponding matrices for method B which contain more elements since the system of Eq. (1) is treated in this case as mathematically coupled. In contrast to method A, the working formula for method B given by Eqs. (3) or (4) depends on all the impulse-response functions of the subsystems contained both in the reference and the target region. In order to calculate this formula the underlying two systems of differential equations associated to the pair of compartmental topologies particularly considered for the reference and target region have to be at the same time analytically solvable. This acts as a constraint on the choice of the desired compartmental topology (i.e. on the total number of compartments) for both tissues (reference and target), i.e. only compartmental topologies which are analytically solvable can be considered. Additionally the terms of the sum in both the nominator (target region) and denominator (reference region) of the inverse Laplace-transformed expression in Eqs. (3) and (4) can be mathematically arbitrarily distributed either to the blood or tissue partial volume. This will bring arbitrariness in the identification of the model parameters with the true dynamic parameters of the kinetic processes. In contrast method A allows the freedom in the choice of the compartmental topology for both reference and target since the two systems of differential equations are solved independently of each other with no constraint about analytical solvability of any of them. In fact it plays no role how the two decoupled systems are solved independently; what matters is that the FIAP time signal Sₚ(t) used further on as input for the target region can be obtained by decomposition of the total time signal into components, namely besides FIAP also the amount of trapped imaging agent in metabolites and the amount of imaging agent within the nonspecific binding subsystems of the reference region including the blood fractional volume. Finally, method A permits also a better identifiability of the compartmental topology for both tissues under study and thus a reliable determination of the dynamic parameters from the model parameters for given topologies. If the decomposition is not possible but the unperturbed dynamic parameters can be at least estimated from some alternative analysis of the kinetics in the reference region (e.g. a graphical method), then method B can be applied (see further observations in sections 2 and 3 below).

The general kinetic analysis for the target region is achieved by the following procedure which is illustrated in FIGS. 4 (method A), 5 (method B), and 6.
1. Data acquisition: Readout of the input data (dynamic time series from target region, Sₜₐᵣ(t), and reference region, S_{ref}(t)) from a medical imaging device 1, for example a PET-scanner.
2. Selection of the appropriate analysis (dynamic or alternatively graphical) of the reference tissue time signal curve S_{ref}(t) (method A and B) in order to obtain the unperturbed dynamic parameters (alternatively steady state parameters like distribution volumes within the VOI are obtained from the graphical analysis) which are then used as initial values when solving the dynamic analysis of the target tissue:
   a. For method A this is visualized in the flowchart of FIG. 4 as follows: First a compartmental topology (system of differential equations) is selected from a list containing multiple alternatives (panel 3 “Reference Tissue Compartmental Model”) which is then solved (panel 5 “SOLVER”) analytically or numerically using appropriate boundary conditions (panels 6 “Boundary Condition” and 7 “Analytical or Numerical Solution of S-ODE's and of Jacobian”). If necessary, further measurements of data like the total concentration of imaging agent in blood (e.g. obtained from image data that lie entirely inside a blood vessel) may be used. The analytical solution (if existing) is selected from a predefined list containing all analytical solutions for the compartmental topologies considered in a “Reference Tissue Compartmental”-library. A particular general compartmental topology for the reference region including fractional blood volume and metabolites is presented in FIGS. 2 and 3. The “Switch” T [symbol] R is set everywhere in the chart on “R” (reference region). The simulation is then fitted to the data (panel 9 “Nonlinear Fit—Optimization”; for a particular embodiment see FIG. 6). The set of unperturbed parameters obtained as output is used to decompose the total time signal from the reference region in order to obtain the free amount of imaging agent in the plasma (panels 11 “All unperturbed Dynamic Parameters” and 12 “Free imaging agent in Plasma”) and finally is taken as input for the initial values of the dynamic parameters for the target region analysis. The FIAP is considered as input function in the target region compartmental analysis. The decomposition of the total signal reveals also the amount of imaging agent either as metabolites or unspecifically bound in both tissue or blood volume fractions of the reference region (see panel 10 “OUTPUT” with subpanels 13 “Blood Elements”, 14 “Metabolites”, 15 “Reference Tissue Unspecific Binding”, and 16 “Modeled Detection Reference Region Signal”).
   b. The flowchart for method B (FIG. 5) is with respect to the analysis of the reference region similar to the flowchart of FIG. 4 described for method A except that alternatively to the dynamic analysis (FIG. 5, panel 7b “Analytical or Numerical Solution of S-ODE's and of Jacobian”) the graphical method can be applied in order to obtain directly the set of unperturbed steady state dynamic parameters (e.g. distribution volumes) describing the kinetics in the reference region (see FIG. 5, panels 12 “steady state unperturbed parameters”, 7c “Graphical Analysis—Linear Fit”). Again this set of data is further on considered as input for the dynamic parameters used in the analysis of the target region.
3. For the dynamic analysis of the target region (see FIGS. 4 and 5, panel 5 “SOLVER” and panel 9 “Nonlinear Fit and/or Optimization”) it must be distinguished between the two possible methods A and B as follows:
   a. If method A is considered (FIG. 4), similar to the case of the reference region (section 2a) one has first to select for the simulation a certain compartmental topology from a list of alternative compartmental models (panel 2 “Target Region Compartmental Model”). Then the model parameters have to be specified (see panel 8 “Initial Values and Boundary Conditions”) and the underlying system of differential equations associated to the selected compartmental model has to be solved analytically or numerically (panels 6 “Boundary Condition” and 7 “Analytical or Numerical Solution of S-ODE's and of Jacobian”). As stated above, the FIAP (panel 12) and the unperturbed dynamic parameters (panel 11) obtained from the analysis of the reference region are considered as input function and initial values of the dynamic parameters, respectively, in the target region compartmental analysis (see FIG. 4 panel 4 “INPUT for Target Region”). The analytical solution (if existing) is selected from a predefined list containing all analytical solutions for the compartmental topologies considered in a “Target Region Compartmental”-library. A particular general compartmental topology for the target region including fractional blood volume and metabolites is presented in FIG. 1. The “Switch” T [symbol] R is set at this level of the analysis everywhere in the flowchart on “T” (target region).
   b. The flowchart for method B (FIG. 5) is similar to the chart described above for method A (FIG. 4) except that the model library (panel 2 “Composite Compartmental Model”) should contain composite compartmental topologies as shown in FIGS. 2 and 3 in which both the target and reference regions are included and contain also fractional blood volume and metabolites. This implies a selection of a particular expression from a list containing multiple alternatives of the functions from Eq. (3) and (4) calculated before the inverse Laplace-transformation is applied for various pairs of compartmental topologies considered in the library described above. Then similar to method A the model parameters have to be specified (initial values and boundary conditions) and the inverse Laplace-transformation of the selected expression has to be performed either analytically or numerically (see FIG. 5, panels 5 “SOLVER”, 7a “Analytical/Numerical Inverse Laplace Trafo and Jacobian”). Finally the Jacobian matrix should be determined. The total time signal and the unperturbed dynamic or steady state parameters of the reference region are considered as input function or possible initial values of the dynamic parameters, respectively, for the analysis of the target region (see FIG. 5, panel 4 “INPUT For Target Tissue”). If existing, the analytical expression of the inverse Laplace-transformation from Eqs. (3) and (4) is selected from a predefined list containing a library with various expressions corresponding to the compartmental topologies considered in the “Composite Compartmental Modal”-library.
4. The simulated total time signal S(t) (obtained by method A or B) is then fitted to the data (see FIGS. 4 and 5, panel 9 “Nonlinear Fit and/or Optimization”) in order to obtain an optimized solution with respect to the relevant parameters (specified under 3a and 3b). A particular embodiment of the “Nonlinear Fit and/or Optimization” is shown in FIG. 6. The optimization method should be a weighted least squares nonlinear fit of the calculated total time signal to the input data from the same VOI. The appropriate algorithm is selected from a list of various alternative algorithms like Levenberg-Marquard, Gauss-Newton, Simplex etc. (see panels 9a “Nonlinear Fit and/or Optimization”, 9b “Perturbation of Dynamic Parameters”, 9c “Simulated Signal” in FIG. 6). The dynamic parameters have to be optimized in order to become independent of their initial values. Appropriate criteria for optimization like χ²/d.o.f., Akaike- and/or F-test etc. should be available for selection from a dedicated library. In order to refine the numerical analysis, the compartmental topology of the system under study can be also numerically determined (identified). In this case various compartmental topologies are analyzed in order to obtain for the error estimation of the dynamic parameters the best score for appropriate test algorithms as for example to minimize χ²/d.o.f This can be applied especially in method A where the analysis of the reference and target regions are performed independently so that also the numerical identification of the compartmental topology can be performed for the reference and target regions independently.
5. At the end of the flowchart (cf. FIG. 4 and 5, panel 110 “OUTPUT”) all the dynamic parameters of the target region are determined (for the optimum compartmental topology) and the simulation of the detection—total time signal curves, i.e. the time dependence of the total amount of imaging tracer within the target region containing contributions from all the subsystems, is performed. Additionally in case when method A is applied the concentration of the imaging agent in all the compartments and/or subsystems of the identified topology within the target region are determined.
   In summary, the final results of this procedure for analysis are
   a. To obtain parametric maps of all relevant dynamic parameters describing the kinetics of the imaging agent within the target region.
   b. For method A, the amount of imaging agent specifically bound, trapped as metabolized products (metabolites), or unspecifically bound in both tissue or blood volume fraction of the target region (see FIG. 4, panel 110 OUTPUT, subpanels 111 “All Pertubed Dynamic Parameters”, 113 “Blood Elements”, 114 “Metabolites”, 115 “Tissue Unspecifically Bound”, 117 “Tissue Specifically Bound”, and 116 “Modeled Detection Signal”) are presented either as parametric maps (regional or on a per-voxel basis) or as resulting time depending model curves (for a given Vol).
   c. Comparison of the output results for reference and target region in order to quantify the perturbation effect due to the presence of detectable metabolites and of the imaging agent specific bound within the target region (see FIG. 4 and 5, panel 18 “Perturbation”). This gives a measure on how the perturbation affects the free and unspecific binding subsystems within the target region relative to the corresponding subsystems within the reference region. This information permits:
      i. The calibration of the data from the target region if the data from reference region are a priori known in absolute units.
      ii. The development of an iterative numerical procedure to decide if a selected VOI fulfills the assumptions needed to be considered as reference region. If the assumptions are not fulfilled, the data will be either impossible to fit with the methods (A and B) described in section 3 or the result of that fit will be physiologically unacceptable. The location and/or size of the VOI are then altered in the next iteration step. This concept can be applied within a validation procedure which self-consistently verifies the perturbation approach for a given VOI.
   d. To obtain the parameter error estimates and all the statistical information (correlation matrix) from the final result of the optimization.
6. Depending on the obtained results about the kinetic of the imaging agent transport within the tissue under study (i.e. from the comparison of the simulations with the time scans) an appropriate development toolkit can be developed (see FIGS. 4 and 5, panel 17 “Clinical Protocols”) to obtain efficient clinical protocols (e.g. schedules for injections or taking image data).

In conclusion, the invention relates to the estimation of kinetic parameters for a target region in the absence of a plasma input function. The invention describes a novel compartmental analysis for time series of image data acquired during a medical procedure utilizing information from a normal (unperturbed) reference region. The proposed composite model models metabolic pathways for blood, reference and target tissue. The proposed analysis procedure features two alternatives:
A Extraction of the plasma input in the reference tissue and its unperturbed kinetic parameters, hereafter utilization of the plasma input and the kinetic parameters of the reference region as initial parameters for the analysis of the target.
B Using the response of the unperturbed reference tissue as an input function for the analysis of the target region. The system of kinetic equations describing the kinetics of the imaging agent in the target and reference region is solved for the input function (amount of free imaging agent in the plasma or the total imaging agent (free and metabolized) contained in plasma, blood elements and metabolites) considered to be the same for both regions.

Further aspects and prerequisites of the invention are:
Different types of detection (MR, CT, PET, SPECT and US) and choices of the applied (smart) imaging agent are possible.
The analysis adapts easily into the clinical workflow, allowing the extraction of the relevant dynamic parameters of the examination on a per- voxel basis and visualizing them as parametric maps, which can be fused with additional (e.g. anatomical) information to improve diagnosis and resulting treatment.
Existing reference tissue concepts are extended within a more general framework including labeled metabolites within tissue and blood. Also the penetration of the metabolites through either the blood-tissue barrier within tissues which can bind (specific and/or nonspecific) the imaging agent or within blood elements is allowed.
The metabolized imaging agent considered can be specifically and nonspecifically bound to non-diffusible binding sites within the tissue and the diffusible which leaves the tissue and flows back into the blood being no longer available as free imaging agent. Thus the metabolites within the system should be described by an appropriate trapping subsystem which should contain one or more compartments (e.g. within blood, tissue or reference tissue) which under certain conditions can be mathematically lumped together.
The clearance of the labeled metabolites out of the body either from the blood metabolite pool or directly from the tissues under study is considered (see FIGS. 1-3, “Exit” 700).
All types of metabolites can flow in and out the reference tissue but they cannot be bound within. Also no metabolization of the free imaging agent within the reference tissue should be allowed. Thus a free movement of the metabolites among all reversible subsystems other then the interstitial ones is not allowed (i.e. the metabolites subsystem has no exchange with any other reversible subsystems except the interstitial one).
The imaging agent will neither be specifically bound nor trapped (metabolized) within the reference tissue volume fraction of the VOI under study.
In method A the dynamic parameters of the imaging agent kinetic within either the blood or the free and/or nonspecific binding tissue volume fractions are determined from the analysis of the reference region whereas in method B the FIAP or the imaging agent in the entire blood volume fraction is expressed (by substitution) in terms of the free and nonspecific binding tissue within the reference tissue VOI.
Adaptability to specific clinical examinations via model selection from various libraries containing multiple alternative models (compartmental topology with eventual the corresponding analytical solution, or appropriate composite general solution) for both reference and target region and specification of model parameters (user interactive).
Visualization of the dynamic parameters as parametric maps and of the imaging agent concentrations in all the compartments of the considered model topology as time signal curves with the possibility to fuse the maps representing functional/morphological information with anatomical information.
Visualization of the parametric map representing the influence of the perturbation (i.e. specific binding within non-diffusible and/or diffusible trapping of imaging agent) on the kinetic of the free and unspecific bound imaging agent within the target region. This involves both the perturbation of the dynamic parameters (transport and metabolism rates) and of the imaging agent concentrations in each subsystem (i.e. compartments for a given compartmental topology) of the target region.

Finally it is pointed out that in the present application the term “comprising” does not exclude other elements or steps, that “a” or “an” does not exclude a plurality, and that a single processor or other unit may fulfill the functions of several means. Moreover, reference signs in the claims shall not be construed as limiting their scope.

1. A data processing system for the evaluation of image data (Sₜₐᵣ(t), S_{ref}(t)) that represent the distribution of at least one imaging agent in a reference region (200) and a target region (210) of a body volume, wherein the system is adapted to evaluate a compartment model of the reference region (200) and the target region (210) with compartments accounting for imaging agent that
(i) is free in blood (300, 1300) or parts thereof;
(ii) is specifically bound (1505) in the target region (210);
(iii) is unspecifically bound in the target region (210), reference region (200) and/or in blood (300, 1300);
(iv) is present in metabolites (600) or other trapping systems.

2. The data processing system according to claim 1, characterized in that it is adapted to evaluate in a first step the compartment model for the reference region (200), and to evaluate in the second step the compartment model for the target region (210) based on the results of the first step.

3. The data processing system according to claim 2, characterized in that the free imaging agent (301) in plasma the determined in the first step and taken as input function for the second step.

4. The data processing system according to claim 2, characterized in that the model parameters calculated in the first step are taken as starting values for the corresponding model parameters in the second step.

5. The data processing system according to claim 1, characterized in that it is adapted to solve the system equations for the target region (210) and the reference region (200) simultaneously based on the assumption that both regions have the same input function.

6. The data processing system according to claim 5, characterized in that the input function is the free imaging agent in plasma (301) or the total imaging agent in blood (300).

7. The data processing system according to claim 1, characterized in that it is adapted to compare the evaluation results for corresponding components of the reference region (200) and the target region (210) in order to validate the correctness of the approach.

8. The data processing system according to claim 1, characterized in that it is adapted to calculate errors associated with the evaluation of the image data based on different compartment models.

9. The data processing system according to claim 1, characterized in that it comprises a display unit for the display of evaluation results.

10. A record carrier on which a computer program for the evaluation of image data that represent the concentration of at least one imaging agent in a reference region (200) and a target region (210) of a body volume is stored, wherein said program is adapted to evaluate a compartment model of the reference region (200) and the target region (210) with compartments accounting for imaging agent that
(i) is free in blood (300, 1300) or parts thereof;
(ii) is specifically bound (1505) in the target region (210);
(iii) is unspecifically bound in the target region (210), reference region (200) and/or in blood (300, 1300);
(iv) is present in metabolites (600) or other trapping systems.

11. Examination apparatus, comprising
an imaging system (1) for the generation of image data, particularly a PET, SPECT, CT, MR, or US system.
a data processing system according to claim 1.

12. A method for the evaluation of image data (Sₜₐᵣ(t), S_{ref}(t)) that represent the distribution of at least one imaging agent in a reference region (200) and a target region (210) of a body volume, comprising the evaluation of a compartment model of the reference region (200) and the target region (210) with compartments accounting for imaging agent that
(i) is free in blood (300, 1300) or parts thereof;
(ii) is specifically bound (1505) in the target region (210);
(iii) is unspecifically bound in the target region (210), reference region (200) and/or in blood (300, 1300);
(iv) is present in metabolites (600) or other trapping systems.
